(19) United States
(12) Patent Application Publication
(10) Pub. No.: US 2016/0068486 A1
(43) Pub. Date: Mar. 10, 2016

(54) PROCESS FOR PREPARING ENANTIOMERICALLY ENRICHED 3-HYDROXYMETHYLPIPERIDINE

(71) Applicant: REUTER CHEMISCHE APPARATEBAU KG, Freiburg (DE)

(72) Inventors: Karl Reuter, Gundelfingen (DE); Tobias Wedel, Karlsruhe (DE); Vasyl Andrushko, Karlsruhe (DE); Christian Wiegand, Freiburg (DE); Florian Stolz, Denzlingen (DE)

(73) Assignee: REUTER CHEMISCHE APPARATEBAU KG, Freiburg (DE)

(21) Appl. No.: 14/785,278

(22) PCT Filed: Apr. 22, 2014
(86) PCT No.: PCT/EP2014/058040

§ 371 Date: Oct. 16, 2015

(30) Foreign Application Priority Data
Apr. 22, 2013 (EP) 13164782.8

(51) Int. Cl. C07D 211/22

(52) U.S. Cl. CPC C07D 211/22

(57) ABSTRACT

The present invention relates to a process for preparing enantiomerically enriched 3-hydroxymethylpiperidine and in particular of the S-enantiomer of (S)-3-hydroxymethyl-piperidine in high chemical and optical purity. The invention also relates to extremely pure (S)-3-hydroxymethylpiperidine and (R)-3-hydroxymethylpiperidine.

The present invention relates to a process for preparing enantiomerically enriched 3-hydroxymethylpiperidine and in particular the S-enantiomer of (S)-3-hydroxymethyl-piperidine in high chemical and optical purity. The invention also relates to extremely pure (S)-3-hydroxymethylpiperidine and (R)-3-hydroxymethylpiperidine.

### BACKGROUND OF THE INVENTION

Both (R)- and in particular (S)-3-hydroxymethylpiperidine are valuable building blocks for the preparation of bioactive compounds, such as antagonistic ligands of receptors in the central nervous system, thrombin inhibitors (see EP 468231), thrombin inhibitors (see WO 99/67215), farnesyltransferase inhibitors (see US 2003/0134846) and vinblastine like antitumor agent desacetyldihydronavelbine (see e.g. Magnus et al., J. Org. Chem., 56 (1991) 1166-1170). As (R)- and (S)-3-hydroxymethylpiperidine are often used at a very early stage in the synthesis of the bioactive compounds, there is need for high amounts of these compounds having high optical and chemical purity.

Several approaches for the preparation of enantiomerically enriched (R)- and (S)-3-hydroxymethylpiperidine have been described in the art. Hereinafter 3-hydroxymethyl-piperidine is termed PPM, while the R- and S-enantiomers thereof are termed (R)-PPM and (S)-PPM, respectively.

For example, Wirz et al., Tetrahedron: Asymmetry (1992), 3(8), 1049-1054 describe resolution of racemic PPM comprising enantioselective hydrolysis with a lipase of the butyryl ester of racemic N-boc protected PPM, yielding N-boc protected (S)-PPM. Deprotection with trifluoroacetic acid followed by extraction with an anion exchange resin, followed by elution with 0.1 NaOH and, extraction with dichloromethane and Kugelrohr distillation of the extract yielded (S)-PPM with an optical purity of 91% ee.

Danieli et al., Tetrahedron: Asymmetry (1996), 7(2), 345-8 describe reduction of S-enantiomer of methyl N-benzyl nipecotate or methyl N-carboxybenzyl nipecotate with lithium tetrahydroborate in tetrahydrofurane followed by removal of the protecting group, however without giving much details on the reaction conditions.

Bettoni et al., Gazzetta Chimica Italiana (1972), 102(3), 189-95 describe the reduction of (S)-N-benzyl nipecotic acid ethyl ester with lithium aluminium hydride in tetrahydrofurane followed by debenzylation to yield (S)-PPM. They also describe direct reduction of (R)-ethyl nipecotate with lithium aluminium hydride in tetrahydrofurane to yield (R)-PPM. Magnus et al., J. Org. Chem., 56 (1991) 1166-1170 describe a similar reaction to obtain (R)-PPM as a yellow oil. The purity of the obtained product is low and during reduction with lithium aluminium hydride a certain loss of the desired enantiomeric excess occurs.

The additional steps for introducing and removing an N-protecting group that are required in the aforementioned 3-hydroxymethylpiperidine synthetic routes are particularly disadvantageous for the preparation of enantiomerically enriched 3-hydroxymethyl piperidine at an industrial scale.

Goswami et al., Organic Progress Research & Development 5 (2001) 415-420 describe fractional crystallisation of the salt of racemic (S)-PPM with L-(−)-dibenzoyl tartaric acid to yield the corresponding salt of (S)-PPM, which was converted directly to (S)-N-(tert-butoxycarbonyl)-3-hydroxymethylpiperidine by hydrolysis and acylation in situ. (S)-3-hydroxymethyl piperidine was not isolated.

The reduction of (R)-nipecotic acid with lithium aluminium hydride (LAH) has been described in WO 2010/027500. Yields of (R)-PPM, however, are low and purity is only moderate. Furthermore, LAH is quite expensive and causes serious safety issues, in particular if it is used at an industrial scale.

Investigations of the inventors of the present invention revealed that the prior art processes do not allow for the preparation of enantiomerically enriched PPM with both high enantiomeric enrichment of at least 98% ee, in particular at least 99% ee and at the same time high chemical purity of at least 98%, in particular at least 99% (determined by gas chromatography).

Therefore, there is still a strong need for providing a process for preparing enantiomerically enriched PPM, that overcomes the disadvantages of the prior art. The process should allow the preparation of enantiomerically enriched PPM with high enantiomeric enrichment of preferably at least 98% ee, in particular at least 99% ee and at the same time high chemical purity, of preferably at least 98%, in particular at least 99% (determined by gas chromatography). Moreover, the process should provide enantiomerically enriched PPM with high yields at low costs.

### SUMMARY OF THE INVENTION

Surprisingly, it has been found that certain boron containing reducing agents, namely BH₃, complexes of BH₃, such as BH₃-ether or thioether complexes, mixtures of tetrahydroborate salt with a metal salt of group 2, 4 or 12 metals and tetrahydroborates of group 2, 4 or 12 metals or lanthanide metals, and mixtures thereof, result in a reduction of enantiomerically enriched piperidine-3-carboxylic acid as well as of esters of enantiomerically enriched piperidine-3-carboxylic acid with high yields and high conservation of stereochemistry without significant formation of organic by-products and, after aqueous work-up, allow for the preparation of enantiomerically enriched PPM with high enantiomeric excess of frequently at least 98% ee, in particular at least 99% ee and at the same time high chemical purity, of preferably at least 98%, in particular at least 99%.

Therefore, according to a first aspect the present invention relates to a process for preparing enantiomerically enriched 3-hydroxymethylpiperidine which comprises the following steps:
a) providing enantiomerically enriched piperidine-3-carboxylic acid or an enantiomerically enriched ester of piperidine-3-carboxylic acid,
b) reduction of the enantiomerically enriched piperidine-3-carboxylic acid or its ester with a boron containing reducing agent to yield a reaction mixture containing enantiomerically enriched 3-hydroxymethylpiperidine; and
c) aqueous work-up of the reaction mixture;
where the boron containing reducing agent is selected from the group consisting of BH₃, complexes of BH₃, mixtures of a tetrahydroborate salt with a metal salt of group 2, 4 or 12 metals and tetrahydroborates of group 2, 4 or 12 metals, and mixtures thereof.

According to a second aspect the present invention relates to non-racemic 3-hydroxymethylpiperidine, which has an enantiomeric excess with regard to one of the enantiomers of 3-hydroxymethylpiperidine of at least 98% ee, in particular at least 99% ee and a chemical purity of at least 98%, in particular at least 99% and especially at least 99.5%, as determined by gas chromatography.

According to a third aspect the present invention relates to a method of extracting 3-hydroxymethylpiperidine from an aqueous alkaline solution, which comprises treatment of an alkaline solution of 3-hydroxymethylpiperidine with an extractant, which is an organic solvent or solvent mixture, where the pH of the aqueous alkaline solution is at least pH 10, in particular at least pH 12.

### DETAILED DESCRIPTION OF THE INVENTION

In the context of the present invention, the terms used generically are defined as follows:
The prefix Cₙ-Cₘ indicates the possible carbon numbers a radical may have.
The term “halogen” as used herein includes e.g. fluorine, chlorine, bromine or iodine, in particular fluorine or chlorine.

The term alkyl relates to a linear or branched alkyl groups having preferably from 1 to 8 carbon atoms (=C₁-C₈-alkyl), in particular 1 to 6 carbon atoms (=C₁-C₆-alkyl), 1 or 4 carbon atoms (=C₁-C₄-alkyl). Examples of C₁-C₄-alkyl include methyl, ethyl, n-propyl, isopropyl, n-butyl, isobutyl and 1,1-dimethylethyl (=tert.-butyl). Examples of C₁-C₈-alkyl include the aforementioned C₁-C₄-alkyl radicals and n-pentyl, 2-pentyl, 2-methylbutyl, 3-methylbutyl, n-hexyl, 2-hexyl, 2-methylpentyl, 3-methylpentyl, 2-ethylbutyl, 2,3-dimethylbutyl, n-heptyl, 2-heptyl, 2-methylhexyl, 3-methylhexyl, 2-ethyl pentyl, 3-ethylpentyl, 2,3-dimethylpentyl, 2,4-dimethylpentyl, n-octyl, 2-octyl, 2-ethylhexyl, etc.

The term “alkoxy” relates to an oxygen bound alkyl radical as defined above having preferably from 1 to 8 carbon atoms (=C₁-C₈-alkoxy), in particular 1 to 6 carbon atoms (=C₁-C₆-alkoxy), 1 or 4 carbon atoms (=C₁-C₄-alkoxy). Examples of alkoxy include in particular methoxy, ethoxy, n-propoxy, isopropoxy, n-butoxy, isobutoxy and 1,1-dimethylethoxy (=tert.-butoxy).

The term “alkoxyalkyl” relates to an alkoxy radical as defined above, which is bound to an alkyl radical, where the alkoxy radical preferably has 1 to 4 carbon atoms and the alkyl part preferably has also 1 to 4 carbon atoms. Examples of alkoxyalkyl include 2-methoxyethyl, 2-ethoxyethyl, 2-propoxyethyl, 2-butoxyethyl, 2-methoxypropyl, 2-ethoxypropyl, 2-propoxypropyl, 3-methoxypropyl, 3-ethoxypropyl, 3-propoxypropyl, 4-metoxybutyl, 3-methoxybutyl, 2-methoxy-2,2-dimethylethyl etc.

The term “aryl” relates to an aromatic or at least partially aromatic mono- or bicyclic hydrocarbon radical, such as phenyl, naphthyl, indanyl, indenyl and tetrahydronaphthyl, which is unsubstituted or may carry 1, 2 or 3 radicals selected from halogen, C₁-C₄-alkyl and C₁-C₄-alkoxy. Aryl is in particular phenyl, which is unsubstituted or may carry 1, 2 or 3 radicals selected from halogen, C₁-C₄-alkyl and C₁-C₄-alkoxy.

The term “arylalkyl” relates to an aryl radical as defined above, in particular to substituted or unsubstituted phenyl, which is bound to an alkyl radical, in particular to a C₁-C₄-alkyl radical. Examples of arylalkyl include benzyl, 1-phenylethyl and 2-phenylethyl, where the phenyl ring in the aforementioned groups is unsubstituted or may carry 1, 2 or 3 radicals selected from halogen, C₁-C₄-alkyl and C₁-C₄-alkoxy.

The term “group 2, 4 or 12 metals” refers to the metals of groups 2, 4 or 12 of the periodic table according to IUPAC and includes in particular the following metals: Mg, Ca, Zr and Zn.

In step a) of the invention, enantiomerically enriched piperidine-3-carboxylic acid or in particular an enantiomerically enriched ester of piperidine-3-carboxylic acid is provided.

Suitable esters of piperidine-3-carboxylic acid include e.g. the alkyl esters of piperidine-3-carboxylic acid, in particular the C₁-C₆-alkyl esters of piperidine-3-carboxylic acid, the alkoxyalkyl esters of piperidine-3-carboxylic acid, e.g. the C₁-C₄-alkoxy-C₁-C₄-alkyl esters of piperidine-3-carboxylic acid, in particular the C₁-C₄-alkoxyethyl esters of piperidine-3-carboxylic acid, arylalkyl esters of piperidine-3-carboxylic acid, e.g. aryl-C₁-C₄-alkyl esters of piperidine-3-carboxylic acid, in particular phenyl-C₁-C₄-alkyl esters of piperidine-3-carboxylic acid such as benzyl or phenethyl esters of piperidine-3-carboxylic acid and aryl esters of piperidine-3-carboxylic acid, e.g. the phenyl ester of piperidine-3-carboxylic acid.

For the purpose of the present invention enantiomerically enriched esters of piperidine-3-carboxylic acid, in particular the C₁-C₆-alkyl esters and benzyl esters of piperidine-3-carboxylic acid, more particularly the C₁-C₃-alkyl esters of piperidine-3-carboxylic acid and especially the ethyl ester of piperidine-3-carboxylic acid are preferred.

The enantiomerically enriched piperidine-3-carboxylic acid as well as the enantiomerically enriched ester of piperidine-3-carboxylic acid which is provided in step a) and reacted in step b) preferably has an enantiomeric excess of at least 80% ee, in particular at least 90% ee. In particular embodiments, an enantiomerically enriched piperidine-3-carboxylic acid or an enantiomerically enriched ester of piperidine-3-carboxylic is used in step b) which has an enantiomeric excess of at least 98% ee, especially at least 99% ee.

Enantiomerically enriched piperidine-3-carboxylic acid as well as enantiomerically enriched esters of piperidine-3-carboxylic acid (the esters are hereinafter termed AEPC), such as the enantiomerically enriched C₁-C₆-alkylester or benzylesters, in particular a C₁-C₃-alkylester and especially the methyl or ethyl ester of piperidine-3-carboxylic acid are known and can be provided by any method known in the art for this or a similar purpose, for example by asymmetric synthesis, by synthesis starting from a chiral precursor, such as enantiomerically enriched piperidine-3-carboxylic acid, or by enantiomeric enrichment of a mixture of the enantiomers of piperidine-3-carboxylic acid or of the enantiomers of the respective piperidine-3-carboxylic acid esters.

Enantiomeric enrichment of piperidine-3-carboxylic acid or of AEPCs can be accomplished by customary methods, e.g. by chiral chromatography or by separation of diastereomers that can be generated by derivatization or salt formation of piperidine-3-carboxylic acid or AEPCs with a chiral resolving agent. Preferred chiral resolving agents in this context are chiral acids capable of forming diastereomeric acid addition salts that can be enriched regarding one enantiomer of the piperidine-3-carboxylic acid or the AEPC, for example by fractional crystallization.

According to a preferred embodiment of the invention step a) of process A comprises subjecting racemic AEPC, in particular a racemic C₁-C₄-alkylester of piperidine-3-carboxylic acid, especially the racemic ethyl ester of piperidine-3-carboxylic acid, to enantiomeric enrichment by fractional crystallization of an acid addition salt of AEPC with a chiral acid. However, it is also possible to enrich non-racemic mixtures of the

AEPC enantiomers in this manner. This enantiomeric enrichment can be used to enrich the R-enantiomer or the S-enantiomer of the AEPC, and is preferably used to enrich the S-enantiomer. Preferred chiral acids in this respect are those known in the art, such as tartaric acid, as described for example in U.S. Pat. No. 5,220,016 and in WO 00/56730, or mandelic acid or dibenzoyl tartaric acid as described in EP1341762, or ethers of 2-hydroxy-propionic acid as described in U.S. Pat. No. 6,340,762. Enantiomeric enrichment of the R-enantiomer of AEPC, in particular of the ethyl ester of R-piperidine-3-carboxylic acid, is preferably achieved by crystallization of the acid addition salt of AEPC with one of the following acids: L(+) tartaric acid or D-mandelic acid. Enantiomeric enrichment of the S-enantiomer of AEPC, in particular the ethyl ester of S-piperidine-3-carboxylic acid, is preferably achieved by crystallization of the acid addition salt of AEPC with one of the following acids: D(−) tartaric acid or L-mandelic acid.

As outlined above, the fractional crystallization of piperidine-3-carboxylic acid or AEPC, respectively, with a chiral acid results in crystals of acid addition salts of piperidine-3-carboxylic acid or AEPC which are enantiomerically enriched with regard to (R)- or (S)-enantiomer. Thus, the mother liquor obtained in said crystallizations is depleted with regard to this respective enantiomer and therefore contains an excess of the opposite enantiomer of piperidine-3-carboxylic acid or AEPC. For example, the mother liquor obtained from the crystallization of (R)-enantiomer acid addition salts is enriched with regard to (S)-enantiomer. In order to avoid loss of yield the piperidine-3-carboxylic acid or APEC, respectively, contained in said mother liquor may be subjected to a racemization. In this way, subsequent to the racemization, an additional amount of the desired enantiomer can be prepared by means of enantiomeric enrichment, for example according to the methods involving fractional crystallization mentioned above. Racemization of non-racemic AEPC is usually accomplished by treating AEPC with a base according to known procedures that are described for example in WO 02/068391. Suitable methods include e.g. treatment with catalytic amounts of sodium ethoxylate as base.

The acid addition salts of piperidine-3-carboxylic acid or AEPC with a chiral acid obtained by the methods for enantiomeric enrichment of the preceding embodiment can be transformed into the free base, i.e. free piperidine-3-carboxylic acid or free AEPC, according to well-known techniques. Typically, for preparing the free base of an AEPC the acid addition salt of the AEPC is treated either with a diluted aqueous base, such as an aqueous solution of an alkali metal carbonate, alkali metal hydrogen carbonate or alkali metal hydroxide such as sodium carbonate, potassium carbonate, sodium hydrogencarbonate, sodium hydroxide, calcium hydroxide or potassium hydroxide, or with a basic ion exchange resin. The free base may be extracted from the thus obtained mixture by a suitable method, such as extraction with an organic solvent. The addition of base is preferably conducted under cooling. It is further preferred to use a concentrated aqueous solution of a base. Typically, for preparing the free base of piperidine-3-carboxylic acid, a solution of its acid addition salt is treated with a basic ion-exchange resin or the acid, which has been used as a chiral auxiliary is precipitated. Furthermore, the free base of piperidine-3-carboxylic acid can be prepared by hydrolysis of an AEPC according to well known methods.

In step b) of the inventive process, the enantiomerically enriched piperidine-3-carboxylic acid or enantiomerically enriched AEPC is subjected to a reduction with a boron containing reducing agent.

According to the present invention, the boron containing reducing agent is selected from the group consisting of the following groups a) to d) and mixtures thereof such as mixtures of the:
a) BH₃, including in situ generated BH₃;
b) BH₃-complexes, such as BH₃-ether or thioether complexes, e.g. BH₃-tetrahydrofurane, BH₃-diethylether or BH₃-dimethyl sulphide adducts,
c) mixtures of a tetrahydroborate salt with a metal salt of group 2, 4 or 12 metals, and
d) tetrahydroborates of group 2, 4 or 12 metals, such as zinc tetrahydroborate.

The enantiomerically enriched piperidine-3-carboxylic acid or AEPC is typically employed in step b) as is, i.e. as the free base, but it may also be used as its acid addition salt, in particular as its monohydrochloride. The enantiomerically enriched AEPC is preferably employed as the free base.

According to another preferred embodiment a) the reducing agent is BH₃, in particular in-situ generated BH₃. In-situ generation of BH₃ can be achieved by well no known techniques such as described by Abiko et al. Tetrahedron Letters (1992), 33(38), 5517-5518; McKennon et al. J.Org. Chem. (1993), 58, 3568-2571; and Prasad et al. Tetrahedron (1992), 48(22), 4623-4628.

Generally, in-situ generation of BH₃ can be achieved by using mixtures of a tetrahydroborate salt, in particular an alkalimetal tetrahydroborate, especially lithium, sodium or potassium tetrahydroborate, with a strong Broenstedt acid such as an organosulfonic acid, e.g. methane sulfonic acid, trifluoromethane sulfonic acid or toluene sulfonic acid or a mineral acid such as H₂SO₄, H₃PO₄ or HCl, or with a Lewis acid such as trimethylsilyl halides, boron halides such as BF₃ or BF₃-etherate. In-situ generation of BH₃ can also be achieved by using mixtures of tetrahydroborate salt, in particular an alkalimetal tetrahydroborate, especially lithium, sodium or potassium tetrahydroborate, with an electrophile such as iodine. In this embodiment a), the relative molar amount of the activating agent (acid or electrophile such as iodine) to the tetrahydroborate may vary and is preferably in the range from 0.05 to 2 mol, in particular from 0.1 to 1.2 mol and especially 0.2 to 1.1 mol per mol of tetrahydroborate.

According to another preferred embodiment c) the reducing agent is selected from mixtures of a tetrahydroborate salt with a metal salt of group 2, 4 or 12 metals. In this context suitable metal salts include the halides, in particular the chlorides, the sulfates, phosphates, oxides, hydroxides, carbonates, C₁-C₁₀-carboxylates and C₁-C₈-alcoholates of these metals. In this context, particularly suitable alcoholates are the alcoholates which are derived from alkanols having 1 to 8 carbon atoms, in particular 1 to 6 or 1 to 4 carbon atoms, such as the methanolates, ethanolates and insopropanolates. In this context, particularly suitable carboxylates are the carboxylates which are derived from aliphatic or aromatic carboxylic acids, in particular mono- or dicarboxylic acids having 1 to 10 carbon atoms, e.g. the formiates, acetates, propionates, butyrates, hexanoates, 2-ethylhexanoates and benzoates.

Preferred salts are the halides and especially the chlorides of these metals.

In embodiment c), suitable tetrahydroborate salts are alkalimetal tetrahydroborates, in particular sodium or potassium tetrahydroborate, and tetra-C₁-C₄-alkylammonium tetrahydroborate such as commercially available tetrabutylammonium tetrahydroborate and also lithium tetrahydroborate.

Particularly suitable metal salts of group 2, 4 or 12 metals are the salts of the following metals: calcium, magnesium, zirconium and zinc, with most preference given to zinc. Particularly preferred salts of the aforementioned metals are the halides, in particular the chlorides.

Particular preference is given to the mixtures of an alkalimetal tetrahydroborate or a tetra-C₁-C₄-alkylammonium tetrahydroborate, in particular sodium or potassium tetrahydroborate or lithium tetrahydroborate, with metal halides, especially metal chlorides, or metal oxides of group 2, 4, 10 or 12 metals, in particular to the mixtures of the zinc chloride, magnesium chloride or calcium chloride, which are preferably in their anhydrous form, such as ZnCl₂, MgCl₂, CaCl₂, ZrCl₄, with particular preference given to zinc halides, especially ZnCl₂.

In this embodiment c), the relative molar amount of the metal salt to the tetrahydroborate salt may vary and is preferably in the range from 0.05 to 2 mol, in particular from 0.1 to 1.2 mol and especially 0.4 to 0.6 mol of metal salt per mol of tetrahydroborate.

The amount of reducing agent will depend on the type of reducing agent in a known manner or can be determined by routine experiments.

If an AEPC is used in embodiment c), the amount of reducing agent will generally be in the range from at least 2 moles of boron bound hydrogen atoms in the boron compound of the reducing agent per mole of enantiomerically enriched AEPC, preferably from 3 to 10 moles, especially from 3 to 8 moles of boron bound hydrogen atoms per mole of enantiomerically enriched AEPC. In particular the amount of reducing agent will be in the range from at least 0.75 moles of boron compound of the reducing agent per mole of enantiomerically enriched AEPC, preferably from 0.75 to 2.5 moles, especially from 0.75 to 2 moles of boron compound per mole of enantiomerically enriched AEPC.

If piperidine-3-carboxylic acid is used in embodiment c), the amount of reducing agent will generally be in the range from at least 2 moles of boron bound hydrogen atoms in the boron compound of the reducing agent per mole of enantiomerically enriched piperidine-3-carboxylic acid, e.g. from 3 to 10 moles, especially from 3 to 8 moles of boron bound hydrogen atoms per mole of enantiomerically enriched piperidine-3-carboxylic acid. In particular the amount of reducing agent will be in the range from at least 0.75 moles of boron compound of the reducing agent per mole of enantiomerically enriched AEPC, e.g. from 0.75 to 2.5 moles, especially from 0.75 to 2 moles moles of boron compound per mole of enantiomerically enriched piperidine-3-carboxylic acid.

If in-situ borane is used as reducing agent, the amount of reducing agent will generally be in the range from at least 4 moles of boron bound hydrogen atoms in the boron compound of the reducing agent per mole of enantiomerically enriched AEPC or piperidine-3-carboxylic acid, e.g. from 4 to 16 moles, especially from 8 to 14 moles of boron bound hydrogen atoms per mole of enantiomerically enriched AEPC or piperidine-3-carboxylic acid. In particular the amount of reducing agent will be in the range from at least 2 moles of boron compound of the reducing agent per mole of enantiomerically enriched AEPC or piperidine-3-carboxylic acid, e.g. from 1 to 4 moles, especially from 2 to 3,5 moles of boron compound per mole of enantiomerically enriched AEPC or piperidine-3-carboxylic acid.

The reaction of step b) is usually performed in an organic solvent, in particular an aprotic organic solvent or a solvent mixture containing predominantly an aprotic solvent. Suitable solvents include, but are not limited to ethers, in particular di-C₁-C₄ alkyl ethers such as diethyl ether, di-n-propyl ethers, diisopropyl ethers, methyl-tert.-butyl ether, ethyl-tert.-butyl ether, mono-, di- and tri-C₂-C₄-alkylylene glycol di-C₁-C₄ alkyl ethers such as dimethyl ethyleneglycol, dimethyl diethyleneglycol, triethyleneglycol dimethyl ether, propyleneglycol dimethyl ether, alicyclic ethers such as tetrahydrofurane, dioxane and methyltetrahydrofurane, aromatic ethers such as anisol and hydrocarbons, e.g. alkanes such as pentane, hexanes, heptanes, cycloalkanes such as cyclopentane, cyclohexane, methylcyclohexane and cycloheptane and aromatic hydrocarbons, in particular mono- and di-C₁-C₄-alkylbenzenes such as toluene, xylenes, isopropylbenzene, tert.-butylbenzene, cumene and mixtures thereof. Suitable solvents may also include alcohols, such as C₁-C₅-alkanols, e.g. methanol, ethanol, n-propanol, isopropanol, n-butanol, isobutanol, tert.-butanol or n-pentanol, mono-, di- and tri-C₂-C₄-alkylylene glycols and their mono-C₁-C₄-alkyl ethers, such as ethylene glycol, propylene glycol, ethylendiglycol or ethylenglycolmonomethylether. The amount of alcohols does generally not exceed 30 Vol.-%, in particular 20 Vol.-%, based on the total amount of organic solvent. Preferably, the reaction is performed essentially in the absence of larger amounts of water, i.e. the amount of water does not exceed 5 Vol.-%, based on the total amount of organic solvent.

Preferably step b) is performed in an aprotic solvent including pure aprotic solvents and mixtures of aprotic solvents. In particular the aprotic solvent comprises an ether, in particular a cyclic ether, or a mono-, di- and tri-C₂-C₄-alkylylene glycol di-C₁-C₄ alkyl ether or a mixture of an ether solvent with a hydrocarbon solvent.

The total amount of solvent used in step b) is usually in the range from 100 to 1000 g, preferably in the range from 250 to 800 g and in particular in the range from 350 to 700 g, based on 1 mol of piperidine-3-carboxylic acid or AEPC, respectively.

The reaction temperature necessary in step b) may vary and depend on the type of reducing agent. Usually temperatures in the range of 10 to 100° C. will be suitable for carrying out step b), however even lower or higher temperatures may be applied without loss of yield or loss of optical purity.

Step b) yields a reaction mixture which contains enantiomerically enriched 3-hydroxy-methylpiperidine. Without being bound to theory it is believed that in the reaction mixture obtained in step b) at least a part of the enantiomerically enriched 3-hydroxy-methylpiperidine is present as a boron containing derivative, e.g. a complex with a boron compound, which must be hydrolysed during aqueous workup to allow isolation of the desired product, namely the enantiomerically enriched 3-hydroxymethyl-piperidine.

Aqueous workup of the reaction mixture obtained in step b) in accordance with step c) of the claimed process can principally be performed by mixing the reaction mixture with water, which may contain an acid or a base, followed by isolation of the desired product, e.g. by liquid or solid phase extraction of the desired product from the thus obtained mixture or by crystallization of a suitable salt of the enantiomerically enriched 3-hydroxymethylpiperidine therefrom. During aqueous workup any boron containing organic compounds will be hydrolysed into boric acid or boric acid esters which can be removed prior to isolation of the desired product but which is not necessarily removed.

It has been found beneficial to perform step c) in a manner that pH of the aqueous phase is at most pH 6, e.g. from pH 0 to pH 6, in particular from pH 0 to pH 3 at least during the initial phase of the workup, in order to achieve hydrolysis of the boron containing reducing agent and optionally present boron containing derivatives of 3-hydroxymethylpiperidine. Therefore a particular embodiment of step c) comprises a hydrolysis of the boron containing reducing agent and optionally present boron containing derivatives of 3-hydroxymethylpiperidine at a pH of at most pH 6, e.g. in the range from pH 0 to pH 6, in particular from pH 0 to pH 3 at least during the initial phase of the workup.

Preferably, aqueous workup also includes an extraction step for isolation of the enantiomerically enriched 3-hydroxymethylpiperidine. It has been found beneficial to extract 3-hydroxymethylpiperidine from an aqueous alkaline solution having a pH of at least pH 10, in particular at a pH of at least pH 12, e.g. at a pH in the range from pH 10 to 14, in particular from pH 12 to 14. Therefore, a particular embodiment comprises treatment of an alkaline solution of 3-hydroxymethylpiperidine with an extractant, which is an organic solvent or solvent mixture, where the pH of the aqueous alkaline solution is at least pH 10, in particular at least pH 12, e.g. at a pH in the range from pH 10 to 14, in particular from pH 12 to 14.

If the hydrolysis products are removed prior to extraction, the removal is preferably performed at a pH of at least pH 6, e.g. in the range from pH 6 to pH 12. Removal of the hydrolysis products can be performed by any conventional steps of solid liquid separation, e.g. by filtration or centrifugation.

In a particular embodiment, the enantiomerically enriched 3-hydroxymethylpiperidine is extracted from the aqueous phase by using an organic solvent or solvent mixture as an extractant, which comprises at least one organic solvent having limited water miscibility. Limited water miscibility in terms of solvents means that the solubility in water at 25° C. and 1016 mbar (deinonized water at pH 7) is at most 30 g/100 ml (=300 g/l). Suitable organic solvents having limited water miscibility include but are not limited to aprotic solvents from the group of ethers, in particular the aforementioned di-C₁-C₄-alkyl ethers, such as diethyl ether, diisopropyl ether, methyl tert.butyl ether and ethyl tert.butyl ether, and alkyl substituted cyclic ethers such as methyltetrahydrofurane, hydrocarbons, in particular aromatic hydrocarbons such as toluene and xylenes, C₁-C₆-alkylesters of C₁-C₆-alkanoic acids, in particular the C₁-C₆-alkylesters of acetic acid or propionic acid such as ethyl acetate, propyl acetate, isopropyl acetate, butyl acetate and ethyl propionate, chlorinated hydrocarbons such as dichloromethane and dichloroethane. Preferred aprotic solvents are selected from alkylaromatic solvents, methyltetrahydrofurane and C₁-C₆-alkylesters of C₁-C₆-alkanoic acids.

Suitable organic solvents having limited water miscibility also include n-butanol, 2-butanol, isobutanol, C₅-C₈-alkanols and C₅-C₈-cycloalkanols.

Likewise suitable as an extractant are tetrahydrofurane or tetrahydrofurane containing mixtures of organic solvents, as tetrahydrofurane is usually not completely miscible with the reaction mixture which is subjected to extraction.

For extraction those organic solvents are preferred which have a boiling point at normal pressure in the range from 30 to 110° C.

It has been found beneficial, if the extractant comprises at least one organic solvent selected from the group of C₁-C₈-alkanols, especially C₁-C₅-alkanols, and C₄-C₈-cycloalkanols. Suitable C₁-C₈-alkanols include methanol, ethanol, n-propanol, isopropanol, 1-butanol, 2-butanol, 2-methylpropanol, 1,1-dimethylethanol (tert.butanol), n-pentanol, 3-methylbutan-1-ol (isoamyl alcohol), n-hexanol, 2-hexanol, 3-hexanol, 2-methylpentanol, isohexanol, n-heptanol, 2-ethylhexanol and n-octanol. Suitable C₅-C₈-cycloalkanols include in particular cyclopentanol and cyclohexanol. A skilled person will readily understand that the organic solvent or solvent mixture, which is used as an extractant will usually comprise at least one organic solvent having limited water solubility. Thus, if the extractant contains a C₁-C₃-alkanol or tert.butanol, it will also contain at least one organic solvent having limited water solubility, in particular at least one aprotic solvent.

Apart from that, the extractant is preferably a mixture of at least one alcohol selected from the group of C₁-C₈-alkanols, especially C₁-C₅-alkanols, and C₄-C₈-cycloalkanols and at least one aprotic solvent having limited water-solubility, in particular a mixture of at least one alcohol selected from the group of C₁-C₈-alkanols, especially C₁-C₅-alkanols, and C₄-C₈-cycloalkanols and at least one aprotic organic solvent selected from the group consisting of alkylaromatic solvents, methyltetrahydrofurane and C₁-C₆-alkylesters of C₁-C₆-alkanoic acids. Preferably the relative amount on a volume base of alcohol to aprotic solvent in these mixtures is from 1:20 to 5:1 v/v in particular from 1:10 to 2:1 v/v.

The enantiomerically enriched 3-hydroxymethylpiperidine can be isolated from the extractant by removing the solvent, preferably under reduced pressure, to obtain a solid residue, which may be subjected to further purification, such as distillation, sublimation or recrystallization. Suitable solvents for recrystallization include e.g. methyltetrahydrofurane and tert.-butylmethyl ether. Optionally present inorganic salts may be removed from an anhydrous solution of 3-hydroxymethylpiperidine by filtration prior to the further purification step.

It may also be possible to extract the enantiomerically enriched 3-hydroxymethyl-piperidine by means of an ion exchange resin, in particular a cationic ion-exchange resin in its H⁺ form. Preferably, the aqueous mixture from which the enantiomerically enriched 3-hydroxymethylpiperidine is extracted by means of an ion exchange resin has a pH of at most pH 8, e.g. in the range from pH 4 to pH 8, in particular from pH 5 to pH 8. Preferably, the aqueous phase of the initial workup containing the enantiomerically enriched 3-hydroxymethylpiperidine is conducted through a bed of the ion exchange resin. Thereby the enantiomerically enriched 3-hydroxymethylpiperidine will be adsorbed by ion-exchange resin, while impurities remain in the aqueous phase. The 3-hydroxymethylpiperidine can be eluted from the ion-exchange resin by treatment of the ion exchange resin with a diluted solution of a suitable organic or inorganic acid, in particular by treatment with diluted hydrochloric acid or diluted aqueous sulphuric acid, whereby an aqueous solution of the corresponding salt of 3-hydroxymethyl-piperidine followed by adjusting the pH to alkaline pH, e.g. to pH of at least pH 10, in particular at least pH 11 and subsequent isolation of the 3-hydroxymethylpiperidine from the aqueous solution by extraction. 3-hydroxymethylpiperidine may also be eluted from the ion-exchange resin by treatment of the ion exchange resin with a diluted aqueous solution of a suitable amine, such as ammonium hydroxide or diethylamine.

By the workup of step c) an enantiomerically enriched 3-hydroxymethylpiperidine is obtained, which has both high optical and chemical purity. In particular optical purity is at least 97% ee, especially at least 98% ee as determined by chiral HPLC, provided that the optical purity of the chiral AEPC or piperidine-3-carboxylic acid, respectively, is not lower. Chemical purity is generally at least 90%, especially at least 95%, as determined by gas chromatography.

The thus obtained enantiomerically enriched 3-hydroxymethylpiperidine can be further purified to yield an enantiomerically enriched 3-hydroxymethylpiperidine which has an enantiomeric excess with regard to one of the enantiomers of 3-hydroxymethyl-piperidine of at least 98% ee, in particular at least 99% ee and a chemical purity of at least 98%, in particular at least 99% and especially at least 99.5%, as determined by gas chromatography. So far, it has not been possible to produce enantiomerically enriched 3-hydroxymethylpiperidine, in particular the S-enantiomer having these high chemical and optical purities, even if the enantiomerically enriched 3-hydroxymethyl-piperidine obtained by the prior methods is further purified by conventional purification techniques such distillation or crystallization.

In contrast to prior art the enantiomerically enriched 3-hydroxymethylpiperidine obtained by the process of the invention can be further purified to increase optical and chemical purity. Further purification may be achieved by recrystallization or distillation of the 3-hydroxymethylpiperidine obtained from work-up c). Suitable solvents for recrystallization include e.g. methyltetrahydrofurane and tert.-butylmethyl ether.

The following examples shall illustrate the invention.

### EXAMPLES

As used herein, the following terms are used as defined below unless otherwise indicated:
ee enantiomeric excess
eq equivalent
er enantiomeric ratio
rt room temperature
ENP ethyl 3-piperidinecarboxylate (ethyl nipecotate)
(S)-ENP (S)-ethyl 3-piperidinecarboxylate ((S)-ethyl nipecotate)
NPA 3-piperidinecarboxylic acid (nipecotic acid)
(S)-NPA (S)-3-piperidinecarboxylic acid (nipecotic acid)
(S)-NPA • HCl (S)-nipecotic acid hydrochloride
GC gas chromatography
MeOH methanol
Me-THF 2-methyltetrahydrofuran
LAH lithium aluminum hydride
PPM 3-hydroxymethylpiperidine (3-piperidylmethanol)
(S)-PPM (S)-3-hydroxymethylpiperidine ((S)-3-piperidylmethanol)
TBME methyl tert-butyl ether
THF tetrahydrofuran
TLC thin layer chromatography

The chemical purity was measured via GC on a Shimadzu GC 2010/GC 14B equipped with a Perkin Elmer PE 624 column (30 m×0.53 mm, hydrogen as carrier, 0.5 bar) and a FID detector. The injection temperature was 250° C., the detector temperature was 300° C., and the temperature gradient was 50° C. (2 min), 10° C./min, 240° C. (3 min). The sample concentration was 10 mg/mL methanol; the injection volume was 2.0 μL. The retention time is Rₜ(PPM)=11 min.

The enatiomeric ratio S/R was measured via chiral HPLC after derivatisation with mosher's acid chloride on a Chiralpak AD 250/4.6/10 column with hexane/isopropanol 90:10 as eluent. The detection wavelength was 210 nm. The retention times were: Rₜ(R-PPM)=9 min and Rₜ(S-PPM)=10 min, respectively.

### I Preparation of Starting Materials

### Example 1

### Preparation of (S)-ENP

1.1 Resolution of ENP with D-Tartaric Acid

To a solution of (S)-ENP (75:25 er; 170 kg) in ethanol/water (94:6; 950 kg) was added 160 kg of D-tartaric acid at 30° C. The mixture was cooled to 20° C. and stirred at 20° C. for 7 h. A solid precipitated out that was collected by centrifugation and washed with ethanol to yield 262 kg of (S)-ENP • D-tartaric acid (98:2 er).

(S)-ENP • D-tartaric acid was recrystallized twice from aqueous ethanol to yield 217 kg of (S)-ENP • D-tartaric acid (99% ee).

### 1.2 Isolation of (S)-ENP From its Tartaric Acid Salt

To a well stirred suspension of 153.6 g of (S)-ENP • D-tartaric acid having an optical purity of 99.4:0.6 er in 157 g of xylene (technical mixture) was added dropwise a solution of 69.3 g of technical potassium hydroxide in 78 g of water under cooling. The organic phase was separated; the water phase was extracted successively with 50 g and then 40 g of xylene. The combined organic phase was dried over 10 g of sodium sulfate. Sodium sulfate was filtered off and xylene was removed under reduced pressure to yield the (S)-ENP as a clear liquid having an optical purity of 99.4:0.6 er.

### Example 2

### Preparation of (S)-Ethyl 3-Piperidinecarboxylate Hydrochloride

To a mixture of (S)-ENP (393 g of a 40% solution in toluene; 1 mol ENP, with an optical purity of 99.4:0.6 er) was added water (120 g) and concentrated hydrochloric acid (120 g) under cooling. The mixture was stirred at 60° C. overnight. The mixture was concentrated at 80° C. under reduced pressure until a thick slurry was obtained. To this mixture was added acetone (200 mL) and the mixture was stirred at room temperature for 1 h. The solid was collected by filtration. From the mother liquor a second crop was obtained by concentration and subsequent addition of acetone.
Yield: 157 g (95%)
   II Preparation of (S)-3-Hydroxymethylpiperidine by Reduction of (S)-ENP with a Mixtures of a Tetrahydroborate Salt and ZnCl₂

### Example 3

### Reduction with NaBH₄+ZnCl₂ in THF (0.5 mol ZnCl₂ per 1 mol Sodium Tetrahydroborate)

A mixture of NaBH₄ (113 g; 3 mol), ZnCl₂ (anhydrous, 204 g; 1.5 mol) and THF (900 g) was stirred under argon at 66° C. for 15 min. The greyish mixture was cooled down to 50° C. and (S)-ENP (99.4:0.6 er; 314 g; 2 mol) was added slowly over 4 h. After complete addition, the mixture was stirred at 50° C. for further 15 min. The mixture was cooled to 20° C., quenched by adding slowly 500 mL of 37% aqueous hydrochloric acid while cooling and then stirred overnight until no further gas was evolved. TLC (MeOH/NH₄OH 3:1; ninhydrin) showed complete conversion to 3-hydroxymethylpiperidine.

The reaction mixture was adjusted to pH=14 by adding a 40% aqueous solution of sodium hydroxide. The mixture was extracted 3 times at 50° C. with a mixture of isobutanol/toluene (1:1; in total 5 L). After drying over MgSO₄, the solvent was removed under reduced pressure to yield crude (S)-PPM as a colorless oil which had a chemical purity greater than 98% and an optical purity 99.4:0.6 er.

The obtained crude (S)-PPM was purified by recrystallization from TBME (2 g per g of crude (S)-PPM). (S)-PPM was obtained in a yield greater than 80%, and had a chemical purity greater than 99% and an optical purity of 99.9:0.1 er.

### Example 4

### Reduction with NaBH₄+ZnCl₂ in THF (0.5 mol ZnCl₂ per 1 mol Sodium Tetrahydroborate))

A mixture of NaBH₄ (2.08 g; 55 mmol), ZnCl₂ (anhydrous, 3.75 g; 27.5 mmol) and THF (70 mL) was stirred under argon for 15 min at 66° C. The mixture was cooled down to 50° C. and (S)-ENP (99.4:0.6 er; 7.86 g; 50 mmol) was slowly added. After complete addition the mixture was stirred at 50° C. for further 30 min. The mixture was cooled to 20° C. The reaction was quenched at 20° C. by adding slowly 11 mL of 37% aqueous hydrochloric acid under cooling. After complete addition the mixture was stirred overnight until no further evolution of gas was observed. TLC showed complete reduction to 3-hydroxymethylpiperidine. The mixture was basified by the addition of a 40% aqueous solution of sodium hydroxide to a pH of 14. The mixture was extracted with isobutanol/toluene (1:1; 3×60 mL). The organic phase was dried over MgSO₄ and concentrated under reduced pressure to yield crude (S)-PPM as a solid (5.96 g) which had an optical purity of 99.4:0.6 er and a chemical purity greater than 98%.

### Example 5

### Reduction with NaBH₄+ZnCl₂ in THF (0.09 mol ZnCl₂ per 1 mol of Sodium Tetrahydroborate)

A mixture of NaBH₄ (4.5 g; 120 mmol), ZnCl₂ (anhydrous 1.5 g; 11 mmol) and THF (75 mL) was stirred under argon for 2 h at 45° C. A solution of (S)-ENP (17.3 g; 110 mmol) in THF (60 mL) was added slowly. After complete addition the mixture was stirred overnight at rt. The mixture was heated again to reflux for 5 h. The reaction was quenched at 20° C. by adding slowly methanol under cooling. TLC (MeOH/NH₄OH 3:1; ninhydrin) showed complete conversion to 3-hydroxymethylpiperidine.

### Example 6

### Reduction with KBH₄+ZnCl₂ in THF (0.5 mol ZnCl₂ per 1 mol of Potassium Tetrahydroborate

A mixture of KBH₄ (4.05 g; 75 mmol), ZnCl₂ (anhydrous 5.2 g; 38 mmol) and THF (70 mL) was stirred under argon for 10 min at 66° C. The mixture was cooled down to 50° C. and (S)-ENP (99.4:0.6 er; 7.9 g; 50 mmol) was slowly added. After complete addition the mixture was stirred for further 30 min at 50° C. The mixture was cooled to 20° C. The reaction was quenched at 20° C. by adding slowly 13 mL of 37% aqueous hydrochloric acid under cooling. After complete addition the mixture was stirred until no further evolution of gas was observed. TLC (MeOH/NH₄OH 3:1; ninhydrin) showed complete conversion to PPM. The pH was adjusted to pH=14 by the addition of a 40% aqueous solution of sodium hydroxide. The mixture was extracted 3 times with a mixture of isobutanol/toluene (1:1; in total 180 mL). After drying over MgSO₄, the solvent was removed under reduced pressure to yield (S)-PPM as a colorless solid having a chemical purity greater than 98% and an optical purity of 99.4:0.6 er.

### Example 7

### Reduction with NaBH₄+ZnCl₂ in Dimethoxyethane

A mixture of NaBH₄ (7.57 g; 200 mmol), ZnCl₂ (anhydrous, 13.6 g; 100 mmol) and dimethoxyethane (150 mL) was stirred under argon for 5 min at room temperature and then for 15 min at 50-60° C. (S)-ENP (99.4:0.6 er, 31.4 g; 200 mmol) was added slowly at 60° C. After complete addition the mixture was stirred for 1 h at 60° C. The mixture was cooled to room temperature and quenched by the adding slowly 60 g of conc. hydrochloric acid under cooling. TLC (MeOH/NH₄OH 3:1; ninhydrin) showed complete conversion to PPM.

III Preparation of (S)-3-Hydroxymethylpiperidine by Reduction of (S)-ENP with a Mixtures of a Tetrahydroborate Salt and Various Metal Chlorides

### Example 8

### Reduction with KBH₄+MgCl₂ in THF

To 9.52 g (100 mmol) MgCl₂ (anhydrous) and KBH₄ (5.39 g; 100 mmol) in THF was added (S)-ENP (7.86 g; 50 mmol, optical purity 99.6:0.4 er) and the mixture was heated to reflux. After complete reaction the mixture was quenched by dropwise addition of 4 M aqueous hydrochloric acid (80 mL). The mixture was basified with sodium hydroxide to pH=14. The mixture was filtered over celite and the resulting filtrate was extracted 3 times with n-butanol/toluene (9:11; 50 mL). Concentration under reduced pressured yielded (S)-PPM as a clear oil (yield 64%) which had an optical purity of 97 ee.

### Example 9

### Reduction with NaBH₄+CaCl₂ in THF

A mixture of NaBH₄ (2.84 g; 75 mmol), CaCl₂ (anhydrous, 4.22 g; 38 mmol) and THF (70 mL) was stirred under argon at 66° C. for 15 min. The mixture was cooled down to 50° C. and (S)-ENP (99.4:0.6 er; 7.86 g; 50 mmol) was added over 10 min. After complete addition, the mixture was stirred at 60° C. for further 10 h. The mixture was cooled to 20° C., quenched by adding slowly 13 mL of 37% aqueous hydrochloric acid while cooling and then stirred overnight.
The reaction mixture was adjusted to pH=14 by adding a 40% aqueous solution of sodium hydroxide. The mixture was extracted 3 times at 50° C. with a mixture of isobutanol/toluene (1:1; 3×60 mL). After drying over MgSO₄, the solvent was removed under reduced pressure to yield an oil (2.55 g with an S-PPM content of 43%).
Yield: approx. 19%
Optical yield: 94:6 er;

### Example 10

### Reduction with NaBH₄+ZrCl₄ in THF

A mixture of NaBH₄ (3.87 g; 100 mmol), ZrCl₂ (anhydrous, 5.83 g; 25 mmol) and THF (50 mL) was stirred under argon at 66° C. for 15 min. The mixture was cooled down to 50° C. and (S)-ENP (8.54 g of a 92% ENP/8% toluene solution; 50 mmol; 99.4:0.6 er) was added over 10 min. After complete addition, the mixture was stirred at reflux for further 2 h. The mixture was cooled to 20° C., quenched by adding slowly a mixture of 18 mL of 37% aqueous hydrochloric acid and 18 mL of water while cooling and then stirred overnight.

0 The reaction mixture was adjusted to pH=14 by adding a 40% aqueous solution of sodium hydroxide. The mixture was extracted 3 times at 50° C. with a mixture of isobutanol/toluene (1:1; 3×60 mL). After drying over MgSO₄, the solvent was removed under reduced pressure to yield an oil (7.8 g with an S-PPM content of 34%).
Yield: approx. 46%
Optical yield: 96.5:3.5 er;

### IV Preparation of (S)-3-Hydroxymethylpiperidine by Various Reductions (Comparative Examples)

### Example 11

### (comparative Example): Reduction with LAH

A mixture of LiAlH₄ (3.8 g; 100 mmol) and THF (75 mL) was stirred at 50° C. for 30 min. The suspension was cooled to 20° C. and a solution of (S)-ENP (98.8:1.2 er; 15.7 g, 100 mmol) in toluene was added slowly (30 min). The mixture was heated to 80° C. for 2 h. The mixture was cooled to 0° C. and 7.6 g of water was slowly added under stirring. Then, a saturated aqueous solution of K₂CO₃ (11.4 mL) was slowly added over 30 min. After stirring for 30 min, Celite (10 g) was added. The mixture was filtered over Celite, washed with THF and concentrated under reduced pressure to yield (S)-PPM as a yellow oil (11 g). The oil was distilled (approx. 1 mbar, up to 220° C. bath temperature) to yield 6.2 g (54%) of (S)-PPM as colorless oil, which solidified during cooling. Chemical purity: 88%; optical purity: 92.9:7.1 er

### Example 12

### (Comparative Example): Reduction with LiBH₄

To a suspension of LiCl (10.6 g; 250 mmol) in THF (100 mL) was added KBH₄ (13.5 g; 250 mmol) and the mixture was stirred for 1 h at room temperature. (S)-ENP (17.1 g of a 92% ENP/8% toluene solution; 100 mmol; 99.4:0.6 er) was added and the mixture was stirred in the presence of Ceramic beads (SiLibeads® Type ZY) for 3 d at room temperature. The mixture was acidified by the addition of 20 mL of conc. hydrochloric acid and 120 mL of water to pH <1. The zirconium balls were removed by filtration and the THF was removed from the clear filtrate under reduced pressure. The residue was basified by the addition of a 40% aqueous solution of potassium hydroxide. The mixture was filtered and the filtrate was extracted with isobutanol/toluene (1:1; 3×50 mL). The organic phase was concentrated under reduced pressure to yield PPM as a clear oil (9.6 g; yield: 69%; PPM content 83%). Optical purity: 58:42 er.

### Example 13

### (Comparative Example): Reduction with NaBH₄ (5 eq) in Ethanol Under Reflux

To a stirred suspension of NaBH₄ (9.5 g; 250 mmol) in absolute ethanol (50 g) was added (S)-ENP (8.54 g of a 92% ENP/8% toluene solution; 50 mmol; 99.4:0.6 er) and the mixture was stirred at 70° C. for 6 h. During this stirring period, further absolute EtOH (60 mL) was added to facilitate stirring. The reaction was quenched by the addition of aqueous hydrochloric acid (300 mmol in 60 g of water). The mixture was stirred for further 3 days at room temperature. Most of the solvent was removed under reduced pressure. The residue was basified by the addition of a 40% aqueous solution of potassium hydroxide under cooling. The mixture was extracted with isobutanol/Me-THF(1:1; 3×30 mL). The organic phase was concentrated under reduced pressure to yield PPM as a clear oil (6.5 g; PPM content 51%; yield: 60%). Optical purity: 68:32 er.

### p Example 14

### (Comparative Example): Reduction with NaBH₄ (4 eq) in THF Under Reflux with Slowly Addition of Methanol

To a stirred suspension of NaBH₄ (9.5 g; 250 mmol) in THF (50 mL) was added (S)-ENP (8.54 g of a 92% ENP/8% toluene solution; 50 mmol; 99.4:0.6 er) and the mixture was heated to 70° C. MeOH (50 mL) was added dropwise during 15 min. After 1 h, the reaction cooled to RT and was quenched by the addition of aqueous sat. KHSO₄ solution (50 mL) and conc. hydrochloric acid (5 mL). THF was removed under reduced pressure and the solution was basified by the addition of a 40% aqueous solution of potassium hydroxide. The precipitated K₂SO₄ was filtered off, and the mixture was extracted at 50° C. with isobutanol/Me-THF(1:1; 5×20 mL). The organic phase was dried (MgSO₄), filtered off and concentrated under reduced pressure to yield PPM as a clear oil (3.9 g; yield: 68%). Optical purity: 78:22 er.

V Preparation of (S)-3-Hydroxymethylpiperidine by Reduction of (S)-ENP with in situ Generated BH₃

### Example 15

### Reduction with the Reaction Product of a Tetrahydroborate Salt with Iodine

To a suspension of NaBH₄ (4.73 g; 125 mmol) in THF (50 mL) was added (S)-ENP (99.4:0.6 er; 7.86 g; 50 mmol) and the mixture was cooled to 0° C. Then a solution of iodine (12.7 g; 50 mmol) in THF (20 mL) was added dropwise. The mixture was heated at 50° C. overnight.

To this mixture was added 1 eq. of NaBH₄ (1.89 g; 50 mmol) and iodine (5.1 g; 20 mmol) and the mixture was stirred at 50° C. for 2 h. The mixture was cooled to 20° C. and conc. hydrochloric acid (15 g) was slowly added. The mixture was stirred overnight at room temperature. The residue was basified by the addition of a 40% aqueous solution of sodium hydroxide to a pH of 13. The mixture was extracted with isobutanol/toluene (1:1; 3×60 mL). The organic phase was dried over MgSO₄ and concentrated under reduced pressure to yield (S)-PPM as a solid (6.7 g) which had an optical purity of 97:3 er and a chemical purity of approx. 75%.

### Example 16

### Reduction with the Reaction Product of a Tetrahydroborate Salt with Broenstedt Acid

To a suspension of NaBH₄ (4.73 g; 125 mmol) in THF (50 mL) was added (S)-ENP (99.4:0.6 er; 7.86 g; 50 mmol) and the mixture was cooled to 0° C. Then a solution of conc. H₂SO₄ (6.4 g; 62.5 mmol) in THF (20 mL) was added dropwise. The mixture was heated to 40° C. for 2 h. To this mixture was added 1 eq. of NaBH₄ (1.89 g; 50 mmol) and conc. H₂SO₄ (2.6 g; 25 mmol) and the mixture was stirred at 50° C. for 2 h. The mixture was cooled to 20° C. and conc. hydrochloric acid (6.4 g) was slowly added. The mixture was stirred overnight at room temperature. The residue was basified by the addition of a 40% aqueous solution of sodium hydroxide to a pH of 13. The mixture was extracted with isobutanol/toluene (1:1; 3×60 mL). The organic phase was dried over MgSO₄ and concentrated under reduced pressure to yield (S)-PPM as a solid (4.5 g) which had an optical purity of 99.4:0.6 er; and a chemical purity greater than 95%)

### VI Preparation of (S)-3-Hydroxymethylpiperidine Starting From the Acid Addition Salt of (S)-NPA

### Example 17

### Reduction of (S)-NPA • HCl with NaBH₄+ZnCl₂ in THF

To a suspension of sodium borohydride (7.6 g; 200 mmol) in THF (20 mL) was added a solution of ZnCl₂ (13.6 g; 100 mmol) in THF (50 mL) and the resulting mixture was heated to 66° C. for 30 min. The mixture was cooled to 40° C. and (S)-NPA hydrochloride (16.6 g; 100 mmol, from EXAMPLE 2) was slowly added. The mixture was heated to reflux for 2 h. The mixture was cooled to room temperature and carefully poured onto 32 g of water and 32 g of concentrated hydrochloric acid. The mixture was stirred overnight, basified with sodium hydroxide (40% solution in water) to pH >13, filtered and extracted three times with iso-butanol/toluene (9:11; 50 mL). Concentration under reduced pressured yielded (S)-PPM as a solid (yield: 40%) which had a chemical purity of 97.9% and an optical purity of 99.85:0.15.

### Example 18

### Reduction of (S)-NPA • HCl with NaBH₄+H₂SO₄ in THF

A suspension of sodium borohydride (7.6 g; 200 mmol) and (S)-NPA hydrochloride (16.6 g; 100 mmol, from EXAMPLE 2) in THF (100 mL) was heated for 2 h at 70° C., till the gas evolution ceased. The temperature was lowered to 50° C. and a solution of concentrated sulfuric acid (10.0 g; 100 mmol) in 40 mL of THF was added during 30 min. The suspension was heated to 70° C. for 1 h, then cooled to room temperature and carefully poured onto 20 g of cracked ice and 20 g of concentrated hydrochloric acid. The mixture was stirred overnight, basified with sodium hydroxide (40% solution in water) to pH >12, filtered and extracted three times with iso-butanol/toluene (9:11; 50 mL). Concentration under reduced pressured yielded (S)-PPM as a solid (47% yield), which had a chemical purity of 96% and an optical purity of 99.64:0.36.

### Example 19

### Extraction of PPM From an Aqueous Alkaline Solution

A solution of PPM (5.0 g) in a saturated, aqueous NaCl-solution (20 g) was adjusted to a pH equal or greater than 13 by the addition of an aqueous solution of sodium hydroxide (1 to 2 drops). The conditions for the extraction as well as the yield of PPM after extraction are indicated in Table 1 below.

TABLE 1

| | | Extraction | |
|---|---|---|---|
| Solvents (ratio) | T | (solvent amount) | yield |
| isobutanol/toluene (1:1) | 50° C. | 5 * 2 mL/g PPM | quantitative |
| isobutanol/metyhl-THF (1:1) | 50° C. | 5 * 2 mL/g PPM | quantitative |
| isobutanol/metyhl-THF (1:1) | 20° C. | 5 * 2 mL/g PPM | Approx. |
| | | | 80% |
| isobutanol/isopropyl | 50° C. | 5 * 2 mL/g PPM | quantitative |
| acetate (1:1) | | | |
| n-butanol/isopropyl | 50° C. | 5 * 2 mL/g PPM | quantitative |
| acetate (1:1) | | | |
| n-octanol | 50° C. | 5 * 2 mL/g PPM | >80% (tlc) |
| n-octanol/Me-THF (1:1) | 50° C. | 5 * 2 mL/g PPM | >80% (tlc) |
| isoamylalcohol/Me-THF (1:1) | 50° C. | 5 * 2 mL/g PPM | >80% (tlc) |
| cyclohexanol/Me-THF (1:1) | 50° C. | 5 * 2 mL/g PPM | >80% (tlc) |
| isopropanol/isopropyl | 50° C. | 5 * 2 mL/g PPM | >80% (tlc) |
| acetate (1:3) | | | |
| ethanol/toluene (1:4) | 50° C. | 5 * 2 mL/g PPM | >80% (tlc) |
| methanol/Me-THF (1:9) | 50° C. | 5 * 2 mL/g PPM | quantitative |
| | | | (tlc) |
| THF | 50° C. | 5 * 2 mL/g PPM | >80% (tlc) |
| isobutanol | 50° C. | 5 * 2 mL/g PPM | approx 50% |
| | | | (tlc) |
| Me-THF | 50° C. | 5 * 2 mL/g PPM | Approx. |
| | | | 60% |

TlC.: >80% (tlc); only traces of PPM are detected on tlc in the remaining water phase and in the organic phase of the 5^{th} extraction.
TlC.: quantitative (tlc);
PPM was not detected on tlc in the remaining water phase and in the organic phase of the 5^{th} extraction.

1. A process for preparing enantiomerically enriched 3-hydroxymethylpiperidine, which comprises the following steps:
a) providing enantiomerically enriched piperidine-3-carboxylic acid or an enantiomerically enriched ester of piperidine-3-carboxylic acid,
b) reduction of the enantiomerically enriched piperidine-3-carboxylic acid or its ester with a boron containing reducing agent to yield a reaction mixture containing enantiomerically enriched 3-hydroxymethylpiperidine; and
c) aqueous work-up of the reaction mixture;
where the boron containing reducing agent is selected from the group consisting of BH₃, complexes of BH₃, mixtures of a tetrahydroborate salt with a metal salt of group 2, 4 or 12 metals and tetrahydroborates of group 2, 4 or 12 metals, and mixtures thereof.

2. The process of claim 1, where an ester of piperidine-3-carboxylic acid, in particular the C₁-C₆-alkyl ester, especially ethyl ester of piperidine-3-carboxylic acid is used.

3. The process of claim 1, where the reducing agent is in-situ generated borane.

4. The process of claim 1, where the reducing agent is selected from the group consisting of mixtures of an alkalimetal tetrahydroborate or a tetra-C₁-C₄-alkylammonium tetrahydroborate, in particular lithium, sodium or potassium tetrahydroborate, with a metal salt of group 2, 4 or 12 metals.

5. The process of claim 1, where the reducing agent is selected from the group consisting of mixtures of an alkalimetal tetrahydroborate or a tetra-C₁-C₄-alkylammonium tetrahydroborate, in particular lithium-, sodium or potassium tetrahydroborate, with a zinc salt, in particular a zinc halide, and zinc tetrahydroborate.

6. The process of claim 1, where step b) is performed in an organic aprotic solvent.

7. The process of claim 6, where the aprotic solvent comprises an ether, in particular a cyclic ether, a di-C₁-C₃-alkoxy-C₂-C₄-alkane or a mixture thereof.

8. The process of claim 1, where step a) comprises subjecting piperidine-3-carboxylic acid or an ester thereof to enantiomeric enrichment with regard to the S-enantiomer by fractional crystallization of an acid addition salt of piperidine-3-carboxylic acid or an ester thereof with a chiral acid.

9. The process of claim 8, where step a) comprises subjecting the ethyl ester of piperidine-3-carboxylic acid with tartaric acid or mandelic acid, in particular D-tartaric acid or L-mandelic acid.

10. The process of claim 1, where step c) comprises a hydrolysis of the boron containing reducing agent and optionally present boron containing derivatives of 3-hydroxymethylpiperidine at a pH of at most pH 6.

11. The process of claim 1, where step c) comprises a first step where the boron containing reducing agent and optionally present boron containing derivatives of 3-hydroxymethylpiperidine are hydrolysed at a pH of at most pH 6 which is followed by a second step where the enantiomerically enriched 3-hydroxymethylpiperidine is extracted from the aqueous phase at a pH of at least pH 10.

12. The process of claim 11, where the enantiomerically enriched 3-hydroxymethyl-piperidine is extracted from the aqueous phase by using an extractant, which is an organic solvent or solvent mixture where the organic solvent or solvent mixture comprises at least one organic solvent having a solubility in water of at most 30 g/100 ml (25° C. and 1016 mbar) and/or tetrahydrofurane.

13. Non-racemic 3-hydroxymethylpiperidine, which has an enantiomeric excess with regard to one of the enantiomers of 3-hydroxymethylpiperidine of at least 98% ee, in particular at least 99% ee and a chemical purity of at least 98%, in particular at least 99% and especially at least 99.5%, as determined by gas chromatography.

14. A method of extracting 3-hydroxymethylpiperidine from an aqueous alkaline solution, which comprises treatment of an alkaline solution of 3-hydroxymethylpiperidine with an extractant, which is an organic solvent or solvent mixture, where the pH of the aqueous alkaline solution is at least pH 10, in particular at least pH 12.

15. The method of claim 14, where the extractant comprises at least one organic solvent having a solubility in water of at most 30 g/100 ml (25° C. and 1016 mbar) and/or tetrahydrofurane.

16. The method of claim 15, where at least one solvent of the extractant is a C₁-C₈-alkanol, especially a C₁-C₅-alkanol, or a C₄-C₈-cycloalkanol.

17. The method of claim 15, where the extractant further comprises at least one aprotic organic solvent that is selected from alkylaromatic solvents, methyltetrahydrofurane and C₁-C₆-alkylesters of C₁-C₆-alkanoic acids.
